(19) United States
(12) Patent Application Publication
(10) Pub. No.: US 2023/0309520 A1
(43) Pub. Date: Oct. 5, 2023

(54) AGE-RELATED HEARING LOSS ANIMAL MODEL AND CONSTRUCTION METHOD THEREFOR

(71) Applicant: UNIVERSITY INDUSTRY FOUNDATION, YONSEI UNIVERSITY WONJU CAMPUS, Gangwon-do (KR)

(72) Inventors: Young Joon SEO, Gangwon-do (KR); Dong Jun PARK, Gangwon-do (KR)

(21) Appl. No.: 18/005,798

(22) PCT Filed: Jul. 26, 2021
(86) PCT No.: PCT/KR2021/009621

§ 371 Date: Jan. 17, 2023

(30) Foreign Application Priority Data
Jul. 29, 2020 (KR) 10-2020-0094819
Feb. 9, 2021 (KR) 10-2021-0018410

(51) Int. Cl. A01K 67/027

(52) U.S. Cl. CPC A01K 67/027; A01K 2207/25

(57) ABSTRACT

The present invention relates to an animal model for age-related hearing loss (ARHL) and a method for preparing the same, particularly to a method for preparing an animal model for hearing loss, which comprises feeding a high fat diet to an animal and injecting galactose to the animal in intermittent hypoxia to age and damage the auditory organ in a short period of time, and an animal model prepared by the preparation method.

### TECHNICAL FIELD

The present invention relates to an animal model for age-related hearing loss (ARHL, presbycusis) and a method for preparing the same, particularly to an animal model for hearing loss prepared by feeding a high fat diet (HFD) to a laboratory animal and injecting galactose to the laboratory animal in intermittent hypoxia, and a method for preparing the same.

### BACKGROUND ART

Interest in geriatric diseases that occur as the average life expectancy increases and thus the society enters an aging society is steadily increasing, and as a result, the proportion of medical expenses is rapidly increasing.

Age-related hearing loss (ARHL, presbycusis), one of geriatric diseases, is hearing loss caused by degenerative changes in the body with increasing age, and is the most common sensory disorder and chronic disease occurring in the elderly over 35 years of age. Hearing loss may begin in the early 40s, but the age at which hearing loss occurs in the conversational area around 1000 Hz and it is thus felt to be difficult to hear is mainly between the ages of 50 and 60, and at the age of 60 or more, the ability to perceive sound decreases even in the low frequency area due to factors such as disease, trauma, and degenerative changes. Hearing loss appears in the form of sensorineural hearing loss in the high-pitched range, and it is known that males are more severely affected. Although the exact mechanism of age-related hearing loss is not known, age-related hearing loss may occur by combination of damage by aging caused by mitochondria dysfunction due to oxidative stress and aging such as death of hair cells, degeneration of hair cells in the cochlea and auditory nerves, eating habits, sleeping habits, exposure to noise, smoking, ototoxic drugs such as aminoglycoside-based antibiotics and loop diuretics, and environmental factors such as family history.

Age-related hearing loss has been recognized as an inevitable part of the human aging process, and its overall contribution to the quality of life has been underestimated, but a survey result revealed that the quality of life of the elderly with hearing loss is evaluated as low overall. It has been confirmed that the causes are lack of efforts to detect hearing loss, the elderly does not accept hearing loss positively even though they recognize it, and the elderly recognizes the use of hearing aids to improve hearing loss as inconvenient.

There are no drugs or treatment methods that have been developed as a way to treat advanced age-related hearing loss yet, and the treatment methods that have been developed so far are to detect patients with age-related hearing loss early and adapt them to daily life through hearing rehabilitation using hearing aids and to perform surgical treatment such as cochlear implant in the case of patients with severe bilateral hearing loss for whom hearing aids are not effective.

Most studies on age-related hearing loss are aimed at prevention and treatment, and an animal model for age-related hearing loss in which aging is induced by genetic manipulation and drug injection is used for studies on the treatment of age-related hearing loss by administering antioxidants to C57B6/J mice, gene therapy, local injection of stem cells and the like, but there is no animal model for age-related hearing loss stimulated by intermittent environmental changes to accelerate aging, so the development of a new animal model for age-related hearing loss is required.

Accordingly, the inventors of the present invention have made efforts to prepare an animal model with age-related hearing loss expressed by the environment, and newly discovered that oxidative stress is continuously induced, aging of cells is accelerated in a short period of time, and age-related hearing loss occurs as a result of feeding a high fat diet (HFD) to a laboratory animal and injecting galactose to the laboratory animal in intermittent hypoxia, whereby completed the present invention.

### SUMMARY OF INVENTION

### Technical Problem

The present invention is to provide an animal model for age-related hearing loss that is prepared by increasing oxidative stress in the body of a laboratory animal and stimulating an aging-related factor but not by genetic manipulation or drug injection to induce age-related hearing loss in a short period of time, and a method for preparing the same.

### Solution to Problem

The present invention provides a method for preparing an animal model for age-related hearing loss, which comprises (1) selecting a laboratory animal with normal hearing among non-human animals; (2) introducing the laboratory animal selected in step (1) into a chamber for creating a hypoxic condition; (3) creating an intermittent hypoxic condition in the chamber of step (2); and (4) feeding a high fat diet or injecting galactose to the animal of step (3).

The present invention also provides an animal model for age-related hearing loss prepared according to the method described above.

The present invention also provides a method for screening a drug for age-related hearing loss, which comprises (a) administering a drug candidate substance for age-related hearing loss to the animal model for age-related hearing loss; (b) measuring a hearing threshold of the animal model to which the candidate of step (a) has been administered; and (c) comparing the hearing threshold of the animal model of step (b) with a hearing threshold of an animal model not administered with the candidate substance and selecting the candidate substance as a drug for age-related hearing loss when the hearing threshold is decreased.

### Advantageous Effects of Invention

The method for preparing an animal model for age-related hearing loss according to the present invention and an animal model prepared by the method can be prepared in a shorter period of time and maintained longer than conventional methods, and it is thus possible to provide an animal model for age-related hearing loss and a method for preparing the same that can promote the development of a treatment strategy for age-related hearing loss.

### BRIEF DESCRIPTION OF DRAWINGS

FIG. 1 is a diagram illustrating a method for preparing an animal model for age-related hearing loss according to the present invention.

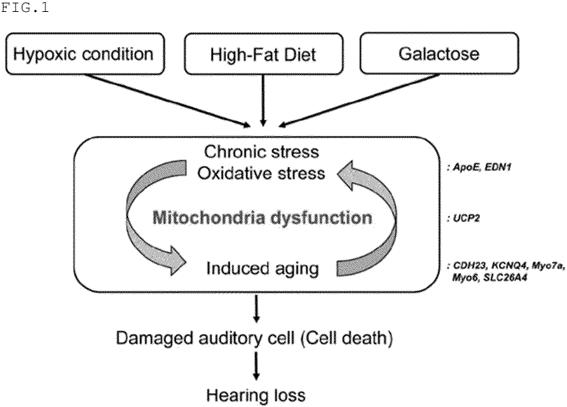

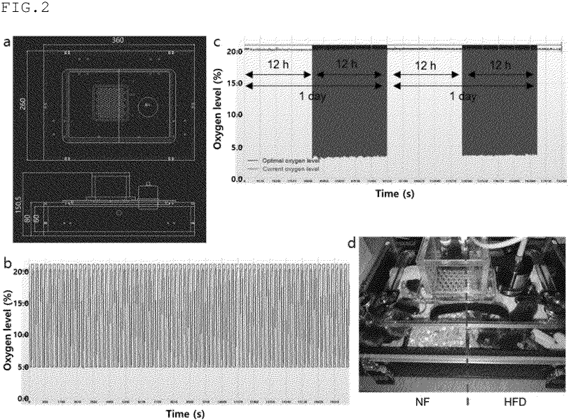

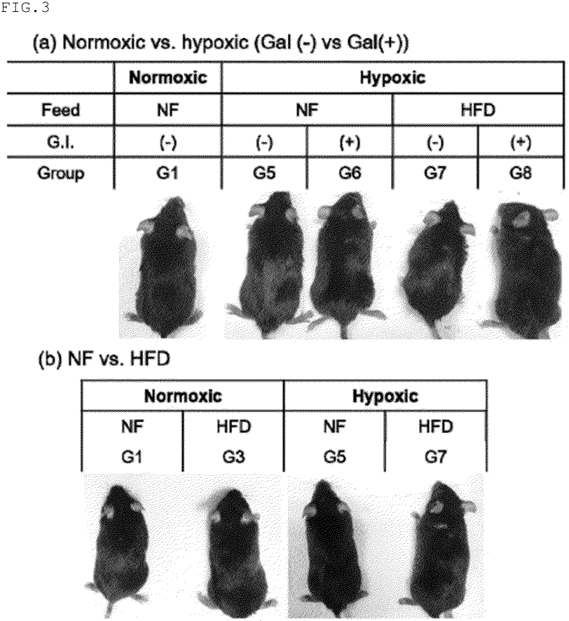

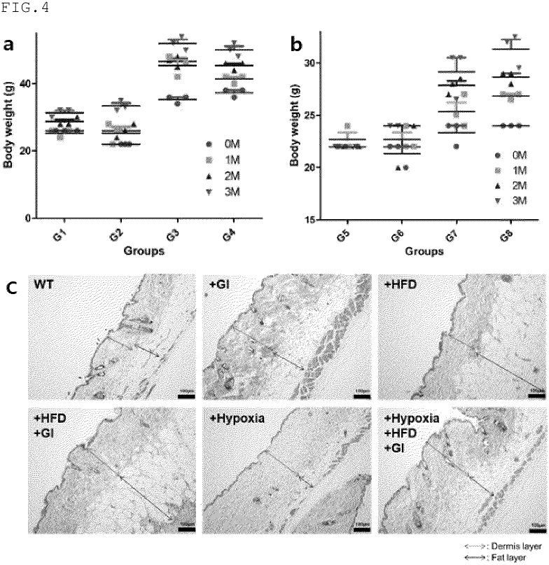

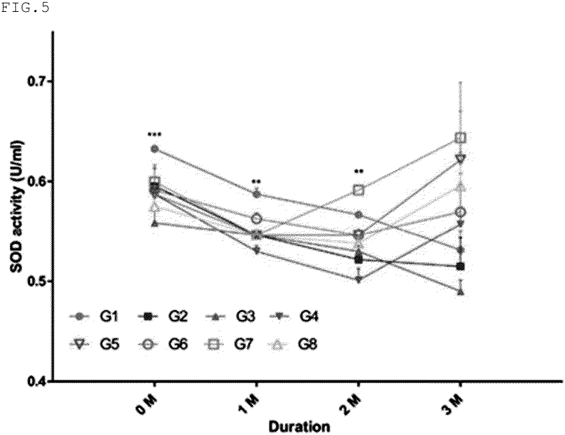

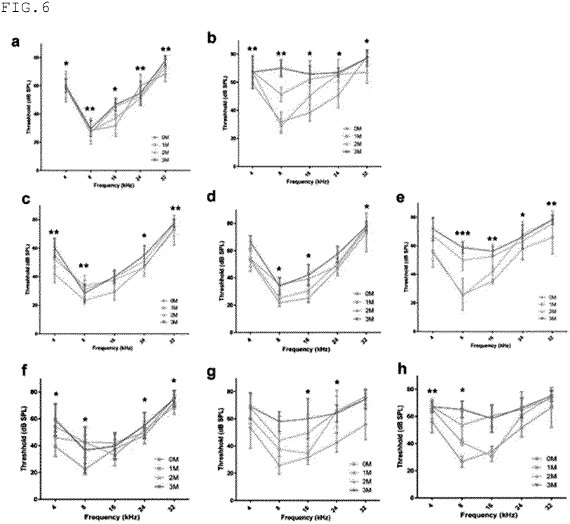

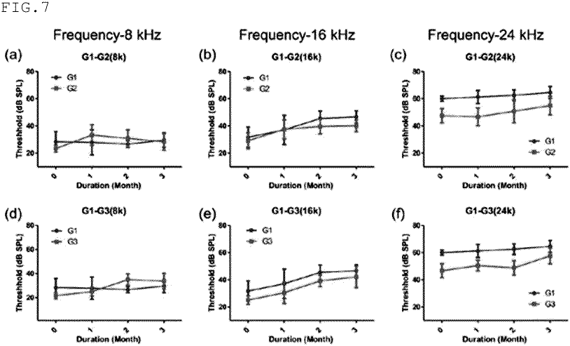

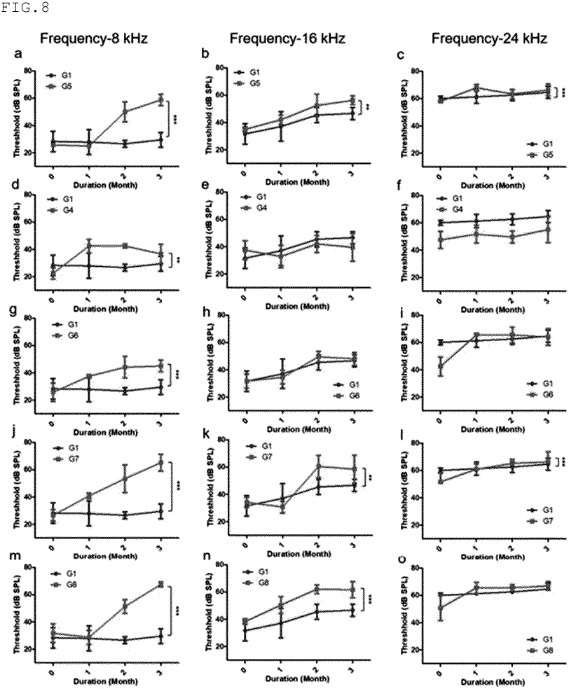

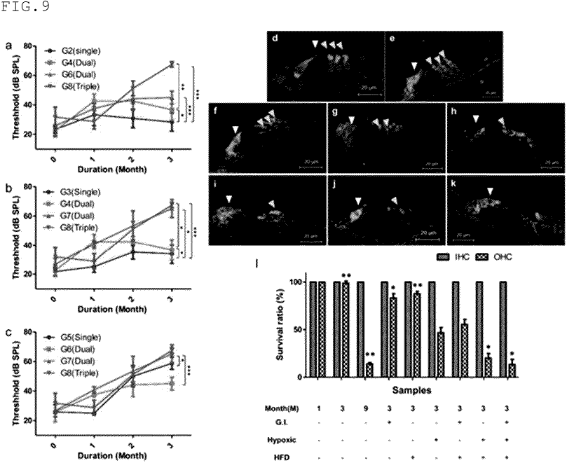

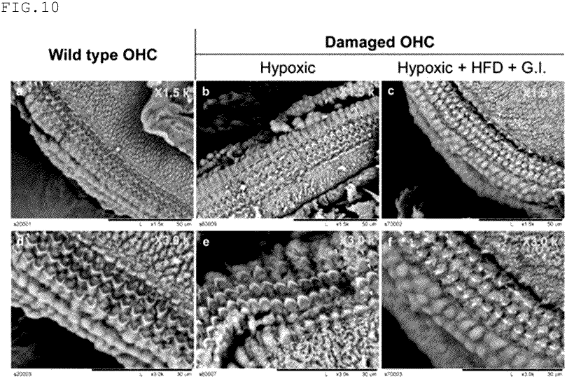

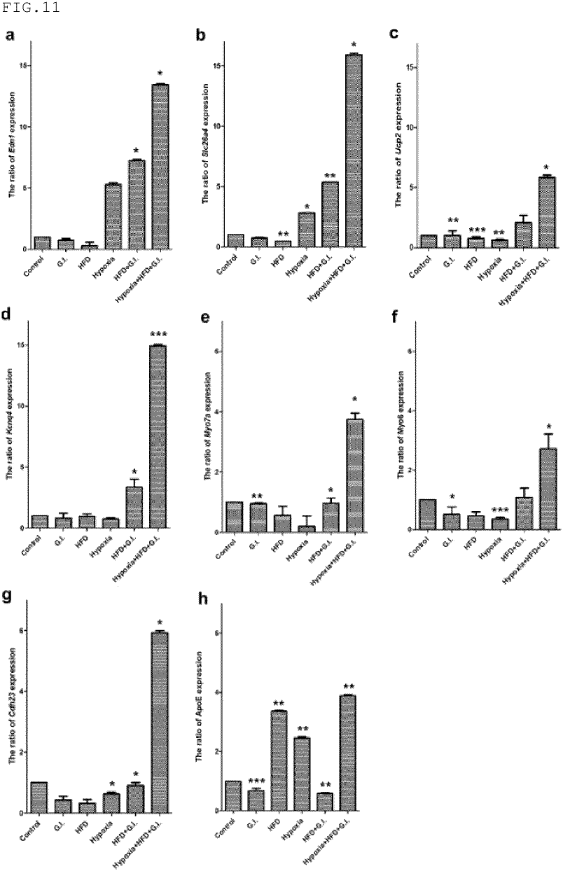

FIG. 2 is a diagram illustrating a hypoxic chamber for intermittent hypoxia of the present invention:
(a) blueprint of hypoxic chamber;
(b) measurement of changes in oxygen level in the hypoxic chamber (blue line: oxygen amount set by the machine; red line: actual oxygen amount);
(c) diagram illustrating the oxygen level in the hypoxic chamber measured every one second for one day; and
(d) actual structure of the hypoxic chamber.

FIG. 3 is a diagram confirming changes in hair color of mice under conditions of hypoxia, high fat diet (HFD) ingestion and/or D-galactose injection (GI) for 12 weeks:
(a) comparison of changes in hair color between normoxic and hypoxic conditions; and
(b) changes in hair color depending on high fat diet ingestion.

FIG. 4 is a diagram confirming changes in body weight, dermal layer thickness and fat layer thickness of mice under conditions of hypoxia, high fat diet ingestion and/or D-galactose injection for 12 weeks:
(a) comparison of changes in body weight under a normoxic condition;
(a) comparison of changes in body weight under a hypoxic condition; and
(c) comparison of changes in skin (dermal layer, skin layer and wrinkles) under hypoxia, HFD or a single, dual or triple condition thereof.

FIG. 5 is a diagram confirming changes in the amount of SOD in the body of mice under conditions of hypoxia, high fat diet ingestion and/or D-galactose injection for 12 weeks:
G1: control; G2: normoxia + normally fed (NF);
G3: normoxia + HFD; G4: normoxia + HFD + GI;
G5: hypoxia + NF; G6: hypoxia + NF + GI;
G7: hypoxia + HFD; and G8: hypoxia + HFD + GI.

FIG. 6 is a diagram confirming changes in hearing threshold of mice under conditions of hypoxia, high fat diet ingestion and/or D-galactose injection for 12 weeks:
(a) control (normoxia and normally fed; G1);
(b) hypoxia, HFD and GI triple condition (G8);
(c) GI single condition (G2);
(d) HFD single condition (G3);
(e) hypoxia single condition (G5);
(f) HFD and GI dual condition (G4);
(g) hypoxia and GI dual condition (G6); and
(h) hypoxia and HFD dual condition (G7).

FIG. 7 is a diagram confirming changes in hearing threshold of mice at various frequencies under a condition of high fat diet ingestion or D-galactose injection for 12 weeks:
(a) comparison of changes in hearing threshold at 8 kHz between G1 and G2;
(b) comparison of changes in hearing threshold at 16 kHz between G1 and G2;
(c) comparison of changes in hearing threshold at 24 kHz between G1 and G2;
(d) comparison of changes in hearing threshold at 8 kHz between G1 and G3;
(e) comparison of changes in hearing threshold at 16 kHz between G1 and G3; and
(f) comparison of changes in hearing threshold at 24 kHz between G1 and G3;

FIG. 8 is a diagram confirming changes in hearing threshold of mice at various frequencies under conditions of hypoxia, high fat diet ingestion and/or D-galactose injection for 12 weeks:
(a) comparison of changes in hearing threshold at 8 kHz between G1 and G5;
(b) comparison of changes in hearing threshold at 16 kHz between G1 and G5;
(c) comparison of changes in hearing threshold at 24 kHz between G1 and G5;
(d) comparison of changes in hearing threshold at 8 kHz between G1 and G4;
(e) comparison of changes in hearing threshold at 16 kHz between G1 and G4;
(f) comparison of changes in hearing threshold at 24 kHz between G1 and G4;
(g) comparison of changes in hearing threshold at 8 kHz between G1 and G6;
(h) comparison of changes in hearing threshold at 16 kHz between G1 and G6;
(i) comparison of changes in hearing threshold at 24 kHz between G1 and G6;
(j) comparison of changes in hearing threshold at 8 kHz between G1 and G7;
(k) comparison of changes in hearing threshold at 16 kHz between G1 and G7;
(l) comparison of changes in hearing threshold at 24 kHz between G1 and G7;
(m) comparison of changes in hearing threshold at 8 kHz between G1 and G8;
(n) comparison of changes in hearing threshold at 16 kHz between G1 and G8; and
(o) comparison of changes in hearing threshold at 24 kHz between G1 and G8;

FIG. 9 is a diagram (a to c) comparing hearing thresholds under single, dual and triple conditions of mice; and a diagram (d to 1) confirming the survival rate of auditory hair cells under conditions of hypoxia, high fat diet ingestion and/or D-galactose injection:
(a) comparison of hearing thresholds under single, dual and triple conditions of GI;
(b) comparison of hearing thresholds under single, dual and triple conditions of HFD;
(c) comparison of hearing thresholds under single, dual and triple conditions of hypoxia;
(d) control (4-week-old mice);
(e) G1 (12-week-old mice; normoxia + NF);
(f) GI single condition;
(g) HFD single condition;
(h) hypoxia single condition;
(i) HFD and GI dual condition;
(j) hypoxia and HFD dual condition;
(k) hypoxia, HFD and GI triple condition; and
(l) survival ratio of outer hair cells (OHC) and inner hair cells (IHC).

FIG. 10 is a diagram confirming the degree of damage to auditory hair cells of mice under conditions of hypoxia, high fat diet ingestion and/or D-galactose injection:
yellow arrow: OHC line; white arrow: IHC line;
red arrow and dotted line: dead or damaged hair cells;
(a) OHC of control (4-week-old mice) (microscopic magnification × 1.5k);
(b) hypoxia (microscopic magnification × 1.5k);
(c) hypoxia + HDF + GI (microscopic magnification × 1.5k);
(a) OHC of control (4-week-old mice) (microscopic magnification × 3.0k);
(e) hypoxia (microscopic magnification × 3.0k); and
(f) hypoxia + HDF + GI (microscopic magnification × 3.0k).

FIG. 11 is a diagram confirming the gene expression effect associated with aging of or damage to the auditory organ of mice under conditions of hypoxia, high fat diet ingestion and/or D-galactose injection:
(a) degree of Edn1 gene expression;
(b) degree of Slc24A4 gene expression;
(c) degree of Ucp2 gene expression;
(d) degree of Kcnq4 gene expression;
(e) degree of Myo7a gene expression;
(f) degree of Myo6 gene expression;
(g) degree of Cdh23 gene expression; and
(h) degree of ApoE gene expression.

### DESCRIPTION OF EMBODIMENTS

Hereinafter, embodiments of the present invention will be described in detail so that those skilled in the art can easily implement the present invention. Embodiments of the present invention are provided to more completely explain the present invention to those skilled in the art. Therefore, embodiments of the present invention may be modified in many different forms, and the scope of the present invention is not limited to the embodiments described below.

Throughout the specification of the present invention, when a part “includes” a certain component, this means that the part does not exclude other components but may further include other components unless specifically stated otherwise.

As used herein, the term “administration” means providing a predetermined substance to a patient by any suitable method, and the route of administration may be intraperitoneal administration, intravenous administration, intramuscular administration, subcutaneous administration, intradermal administration, oral administration, topical administration, intranasal administration, intrapulmonary administration, or intrarectal administration through any general route as long as the substance can reach the target tissue, but is not limited thereto. The composition may be administered in a form mounted on any device capable of transporting the active substance to the target cell.

As used herein, the term “hearing loss” generally means decreases in sensitivity to sound and ability to hear of an individual. “Presbycusis” is hearing loss caused by aging of the auditory organ, and is a symptom in which speech discrimination is reduced due to an increase in the hearing threshold and a decrease in the processing of acoustic stimuli by the central nervous system.

As used herein, the term “hearing threshold” means the smallest sound that can be heard at each frequency.

The present invention provides a method for preparing an animal model for age-related hearing loss, which comprises
(1) selecting a laboratory animal with normal hearing among non-human animals;
(2) introducing the laboratory animal selected in step (1) into a chamber for creating a hypoxic condition;
(3) creating an intermittent hypoxic condition in the chamber of step (2); and
(4) feeding a high fat diet or injecting galactose to the animal of step (3).

The present invention also provides an animal model for age-related hearing loss prepared by the method described above.

Hereinafter, the method for preparing an animal model for age-related hearing loss according to the present invention will be described in detail step by step.

Step is a step of selecting a laboratory animal with normal hearing, and the laboratory animal with normal hearing may be a laboratory animal having a hearing threshold of 30 dB or less, specifically a hearing threshold of 25 dB or less, preferably 20 dB or less. The laboratory animal is not particularly limited as long as it is suitable for evaluating the efficacy of the study on age-related hearing loss, and may be one or more non-human animals selected from the group consisting of rats, mice, rat gerbils, guinea pigs, monkeys, dogs, cats, rabbits, cows, sheep, pigs and goats, preferably rats, mice, or rat gerbils. The raising conditions of laboratory animals before animal models are prepared by the preparation method are not particularly limited and conventional methods may be used, but it is desirable to raise the individuals under the same conditions as much as possible in order to obtain homogeneous laboratory animals.

Step is a step of introducing the laboratory animal into a chamber in which a hypoxic condition can be created, and the chamber may be a hypoxic chamber, which is made of acrylic sheets, can have the oxygen concentration adjusted, and has an internal volume of 3 to 7 L, preferably 3 to 6 L, but is not limited thereto. Since the chamber is divided by a wall in the center, mice under different conditions may be distinguished, and up to 5 mice may be introduced into each chamber.

Step is a step of creating an intermittent hypoxic condition and exposing the laboratory animal to the intermittent hypoxic condition, and is to accelerate cell aging by temporarily blocking the supply of oxygen to the blood. The hypoxia may be created by introducing nitrogen into the chamber to adjust the oxygen concentration, that is, to decrease the oxygen concentration from about 15% to 20% (normoxia) to 10% or less (hypoxia), preferably to 5% or less. The intermittent hypoxia may be created by alternately changing the oxygen concentration from normoxia to hypoxia for 8 to 16 hours, specifically by alternately changing the oxygen concentration from normoxia to hypoxia for 10 to 14 hours, preferably by alternately changing the oxygen concentration from normoxia to hypoxia at intervals of 12 hours.

Step is a step of feeding a high fat diet or injecting galactose to the laboratory animal exposed to an intermittent hypoxic condition, and the fed high fat diet is intended to induce hyperlipidemia in blood vessels and accelerate cell aging due to metabolic abnormalities, and may be a cause of hearing loss due to an imbalance in nutrient supply by a decrease in blood flow by causing hyperlipidemia in the blood vessels of the auditory organ. The high fat diet may be a food containing vitamins and having a fat content of 20% to 60%, specifically 20% to 50%, preferably 25% to 45%.

The injected galactose is intended to cause oxidative stress and mitochondria dysfunction and accelerate cell aging, may be injected at 100 to 700 mg, specifically 200 to 600 mg, preferably 500 mg per kg of body weight of the laboratory animal, and may be injected once or twice a week, but is not limited thereto. The galactose may be injected by a generally known method, preferably subcutaneously using a syringe.

In step (4), feeding of a high fat diet and injection of galactose may be performed in parallel.

In the present invention, the preparation period in the preparation method may be 4 to 16 weeks, specifically 6 to 14 weeks, preferably 8 to 12 weeks. Age-related hearing loss may not be expressed in a case where the preparation period is 3 weeks or less, and hearing loss may be aggravated to lead to complete hearing loss in a case where the preparation period is 17 weeks or more.

In the present invention, the age-related hearing loss may be caused by aging, drinking, genetic predisposition, continuous exposure to noise, vascular or metabolic factors, exposure to ototoxic drugs, and the like, and speech discrimination and sound localization may deteriorate by deterioration in the auditory processing ability of the central nervous system due to aging in a noisy environment.

The age-related hearing loss may be one or more kinds of hearing loss selected from the group consisting of sensory presbycusis due to damage to outer hair cells and supporting cells of the basal turn of the cochlear, neural presbycusis due to damage to the cochlear auditory nerve, metabolic presbycusis (strial presbycusis) due to atrophy of the vasculature of the middle and apical turns of the cochlea, cochlear conductive presbycusis due to reduced mechanical mobility of the cochlea, intermediate presbycusis due to lesions of the central nervous system, and mixed presbycusis due to one or more of the causes.

In the present invention, the preparation method may be a preparation method of an animal model for age-related hearing loss in which hearing loss is expressed due to one or more effects selected from the group consisting of fat accumulation, increased oxidative stress, induction of mitochondria dysfunction, accelerated aging, auditory organ aging and damage-associated gene expression, and auditory hair cell death effect. The effect can be expressed under a single condition of hypoxia, but there is a synergy effect under the dual condition of high fat diet ingestion or D-galactose injection in hypoxia, and there is a remarkably excellent synergy effect under the triple condition of hypoxia, high fat diet ingestion, and D-galactose injection.

The expressed gene that is associated with aging of or damage to the auditory organ may be one or more genes selected from the group consisting of Edn1, a gene associated with cardiovascular complication, Slc24A4, an ion channel-related gene in the auditory organ, Ucp2, a gene associated with mitochondria dysfunction, Kcnq4, Myo7a, and Myo6, genes associated with non-syndromic hearing loss, Cdh23, a gene associated with age-related hearing loss, and ApoE, a gene associated with age-related disorders and vascular diseases.

In the present invention, the age-related hearing loss may be determined as hearing loss when the hearing threshold is 50 dB or more. The hearing threshold is the smallest sound that can be heard within each frequency when a pure tone is heard at each frequency, and hearing loss may be classified into mild hearing loss (hearing threshold: 26 to 40 dB), moderate hearing loss (hearing threshold: 41 to 55 dB), moderately severe hearing loss (hearing threshold: 56 to 70 dB), severe hearing loss (hearing threshold: 71 to 90 dB) and profound hearing loss (hearing threshold: 91 dB or more) depending on the hearing threshold.

The hearing threshold may be determined by conducting an auditory brainstem response test (ABR-TEST). The auditory brainstem response test is a test in which sound stimuli are heard and electrical responses from the auditory system are recorded through electrodes located on the scalp, and the hearing threshold may be determined through the response threshold of V wave, which is recorded loudly and clearly and is used for response analysis.

In the present invention, age-related hearing loss induced by the preparation method may be maintained chronically unlike conventional animal models for hearing loss, specifically for 3 months or more, more specifically for 1 month or more. Age-related hearing loss in conventional animal models for hearing loss is naturally healed over time, thus the time to evaluate drug efficacy is short, it may be difficult to determine the drug efficacy, and the reproducibility may be poor even by the same method. However, in the animal model for age-related hearing loss of the present invention, hair cells are killed and are not regenerated, and thus age-related hearing loss in laboratory animals is not naturally healed but may be maintained chronically.

In a specific embodiment of the present invention, the present inventors have observed the changes of mice to which the preparation method has been applied for 3 months, and as a result, confirmed that there are effects of changes in hair color due to oxidative stress and aging (see FIG. 3); increases in body weight, fat mass and wrinkles (see FIG. 4), oxidative stress (see FIG. 5) and hearing threshold (see FIGS. 6 to 8); damage to and death of auditory hair cells (see FIGS. 9 and 10); and increased expression of genes, Edn1, Slc24A4, Ucp2, Kcnq4, Myo7a, Myo6, Cdh23, and ApoE, associated with aging of or damage to the auditory organ (see FIG. 11), and that age-related hearing loss has been induced in the mice. It has been confirmed that there is a remarkably excellent synergy effect under the dual and triple conditions than under the single conditions.

The present invention also provides a method for screening a drug for age-related hearing loss, which comprises:
(a) administering a drug candidate substance for age-related hearing loss to the animal model for age-related hearing loss prepared above;
(b) measuring a hearing threshold of the animal model to which the candidate substance of step (a) has been administered; and
(c) comparing the hearing threshold of the animal model of step (b) with a hearing threshold of an animal model not administered with the candidate substance, and selecting the candidate substance as a drug for age-related hearing loss when the hearing threshold is decreased.

In the present invention, the hearing threshold, administration method, and kind of age-related hearing loss in the method for screening a drug for age-related hearing loss are the same as those in the description of the method for preparing an animal model for age-related hearing loss, and thus the above contents are used for specific description.

### EXAMPLES

Hereinafter, the present invention will be described in detail by way of Examples.

However, the following Examples are merely illustrative of the present invention, and the contents of the present invention are not limited to the following Examples.

### <Experimental Example 1> Experimental Method Of Animal Model for Age-Related Hearing Loss

### <1-1> Experimental Group

A total of 72 C57BL/6 mice (male, 12 weeks old) were classified into 8 groups according to whether or not they were exposed to three environmental conditions related to the expression of intermittent oxidative stress. Classification criteria and classified groups are as shown in Table 1 below.

TABLE 1

| | Normoxia | | | Hypoxia | |
|---|---|---|---|---|---|
| | Food | GI (D-galactose injection) | | Food | GI |
| G1 | NF | X | G5 | NF | X |
| G2 | NF | O | G6 | NF | O |
| G3 | HDF | X | G7 | HDF | X |
| G4 | HDF | O | G8 | HDF | O |

NF = Normal diet, HFD = High fat diet, and GI = D-galactose injection

### <1-2> Animal Testing Procedure

*After receiving approval from the Animal Care and Use Committee of the Animal Laboratory in Wonju College of Medicine, Yonsei University (approval number: YWC-181001-2), animal experiments were conducted using C57BL/6 mice (male, 12 weeks old). The mice were observed at room temperature with a 12-hour light/dark cycle under various oxygen conditions including normoxia and intermittent hypoxia.

The respective groups were classified into 8 groups as shown in Table 1 above according to exposure to the three environmental conditions. Regarding oxygen concentration, the mice were classified into a normoxic group and a hypoxic group. Specifically, the mice of groups G1 to G4 were raised in normoxia having an oxygen concentration of 20%, and the mice of groups G5 to G8 were raised in hypoxia having an oxygen concentration of 5% for 12 hours/day and in normoxia for 12 hours/day.

Regarding fat ingestion, the mice were classified into a normal diet (NF; NIH-41, autoclaved, Zeigler Bros Inc., PA, USA) group and a high fat diet (HFD; High Fat Diet 32, autoclaved, CLEA Japan Inc., Tokyo, Japan) group. The NF and HFD were prepared and mixed in a Korean plant. Specifically, the mice of groups G3, G4, G7, and G8 ingested HFD with 32% fat content containing vitamins, and the mice of groups G1, G2, G5, and G6 ingested NF. The compositions of HFD and NF are as shown in the following Table 2 (Ingredients of HFD 32), Table 3 (AIN93-VX vitamin mix in HFD 32), Table 4 (AIN93G mineral mix of HFD 32), Table 5 (Ingredients of NIH-41), Table 6 (Amino Acid in NIH-41), Table 7 (Mineral Concentration in NIH-41), Table 8 (Vitamin Concentration in NIH-41) and Table 9 (Analysis and Comparison of Ingredients of HFD 32 and NIH-41), respectively.

TABLE 2

| Ingredient Composition | % per weight |
|---|---|
| Milk casein | 24.5 |
| Egg white | 5 |
| L-cystine | 0.43 |
| Powdered beef tallow (including 80% of beef tallow) | 15.88 |
| Safflower oil (high oleic acid) | 20 |
| Crystalline Cellulose | 5.5 |
| Maltodextrin | 8.25 |
| Lactose | 6.928 |
| Sucrose | 6.75 |
| AIN93 vitamin mix | 1.4 |
| AIN93G mineral mix | 5 |
| Choline bitartrate | 0.36 |
| Teriary butylhydroquinone | 0.002 |
| Total | 100 |

TABLE 3

| Ingredient Composition | g per weight |
|---|---|
| Niacin | 3.0 |
| Calcium Pantothenate | 1.6 |
| Pyridoxine HC1 | 0.7 |
| Thiamin (81%) | 0.6 |
| Riboflavin | 0.6 |
| Folic Acid | 0.2 |
| Biotin | 0.02 |
| Vitamin B 12(0.1 % in mannitol) | 2.5 |
| Vitamin E, DL-alpha tocopheryl acetate (500IU/g) | 15.0 |
| Vitamin A Palmitate (500,000IU/g) | 0.8 |
| Vitamin D3, Cholecalciferol (500,000 IU/g) | 0.2 |
| Vitamin K1, Phylloquinone | 0.075 |
| Sucrose, fine ground | 974.705 |
| Total | 1000 |

TABLE 4

| Ingredient Composition | g per weight |
|---|---|
| Calcium Carbonate | 357.0 |
| Potassium Phosphate | 196.0 |
| Potassium Citrate | 70.78 |
| Sodium Chloride | 74.0 |
| Potassium Sulfate | 46.6 |
| Magnesium Oxide | 24.3 |
| Ferric Citrate | 6.06 |
| Zinc Carbonate | 1.65 |
| Magnanous Carbonate | 0.63 |
| Cupric Carbonate | 0.31 |
| Potassium Lodate | 0.01 |
| Sodium Selenate | 0.0103 |
| Ammonium Paramolybdate | 0.008 |
| Sodium Meta-Silicate | 1.45 |
| Chromium Potassium Sulfate | 0.275 |
| Lithium Chloride | 0.0174 |
| Boric acid | 0.0815 |
| Sodium Fluoride | 0.0635 |
| Nickel Carbonate Hydroxide | 0.0318 |
| Ammonium Meta-vanadate | 0.0066 |
| Sucrose | 220.7159 |
| Total | 1000 |

TABLE 5

| Ingredient (Unit) | % per weight |
|---|---|
| Ground whole wheat | 34.9 |
| Ground No. 2 Yellow Corn | 21.0 |
| Ground whole oats | 10.0 |
| Wheat Middlings | 10.0 |
| Fish Meal (60% protein) | 9.0 |
| Soybean Meal (45% protein) | 5.0 |
| Soy Oil | 2.0 |
| Alfalfa Meal (17% protein) | 2.0 |
| Corn gluten meal (60% protein) | 2.0 |
| Dicalcium phosphate | 1.5 |
| Brewers dried east | 1.0 |
| Premixes | 0.6 |
| Limestone | 0.5 |
| Salt | 0.5 |

TABLE 6

| Amino Acid Concentration | % of total diet |
|---|---|
| Arginine | 0.9 |
| Lysine | 0.85 |
| Methionine | 0.35 |
| Cystine | 0.25 |
| Tryptophan | 0.2 |
| Glycine | 0.95 |
| Histidine | 0.38 |
| Leucin | 1.4 |
| Isoleucine | 0.95 |
| Phenylalanine | 0.85 |
| Tyrosine | 0.6 |
| Threonine | 0.65 |
| Valine | 0.9 |
| Glycine | 0.95 |

TABLE 7

| Mineral Concentration (Unit) | per weight |
|---|---|
| Calcium (%) | 1 |
| Phosphorous (%) | 0.94 |
| Potassium (%) | 0.55 |
| Sodium (%) | 0.25 |
| Magnesium (%) | 0.15 |
| Iron (ppm) | 300 |
| Zinc (ppm) | 40 |
| Manganese (ppm) | 140 |
| Copper (ppm) | 12 |
| Cobalt (ppm) | 0.7 |
| Iodine (ppm) | 1.8 |

TABLE 8

| Vitamin Concentration (Unit) | per weight |
|---|---|
| Vitamin A (IU/g) | 17 |
| Vitamin D3 (IU/g) | 4 |
| Alpha-Tocopherol (IU/g) | 45 |
| Thiamine (ppm) | 15 |
| Riboflavin (ppm) | 9 |
| Niacin (ppm) | 70 |
| Pantothenic Acid (ppm) | 30 |
| Choline (ppm) | 1900 |
| Folic Acid (ppm) | 2 |
| Biotin (ppm) | 2 |
| Vitamin B12 (Mcg/kg) | 75 |
| Vitamin K (ppm) | 2 |

TABLE 9

| Guaranteed Analysis | NIH-41 | HFD32 |
|---|---|---|
| Moisture with vitamins | 36.9 | 6.2 |
| Crude Protein | 18.0 | 25.5 |
| Crude Fat | 5.0 | 32.0 |
| Crude Fiber | 5.0 | 2.9 |
| Ash (%) | 35.1 | 4.0 |
| Nitrogen-free extract (%) | - | 29.4 |
| Total | 100 | 100 |

Regarding galactose injection, the mice were classified according to whether or not they were injected with D-galactose (GI). Specifically, the mice of groups G2, G4, G6, and G8 were subcutaneously injected with D-galactose (G0750, Sigma-Aldrich, USA) dissolved in 0.9% saline at a concentration of 500 mg/Kg, and the mice of groups G1, G3, G5 and G7 were subcutaneously injected with 0.9% saline (vehicle).

The mice were weighed weekly to confirm changes, the auditory brainstem response (ABR) was measured every two weeks to evaluate the hearing threshold, and serum samples were collected monthly to check for oxidative stress in the body.

### <1-3> Design of Hypoxia

A hypoxic chamber was designed to subject mice to oxidative stress.

Specifically, the hypoxic chamber made of acrylic sheets was composed of a fan for automatically introducing fresh air and nitrogen into the upper part and dispersing the air and a wall for dividing the food types into NF groups and HFD groups. The hypoxic chamber (340 × 240 × 60 mm, internal volume: 4.9 L) was designed so as to control the oxygen concentration through automatic nitrogen introduction using an LCI system (Live Cell Instrument Co., Seoul, Korea) (see FIG. 2).

With a total of 24 hours reaction as one cycle, nitrogen was automatically introduced every 2 minutes for 12 hours to lower the oxygen concentration to 5%, sufficient oxygen was supplied by introducing fresh air after 12 hours, and the oxygen concentration was recorded every one second every day (see b and c of FIG. 2). All gases used were discharged out of the building through tubes, and the amount of oxygen in the chamber was checked using an oxygen indicator. Cleaning was performed once a week to remove food, water and feces of mice.

### <1-4> Auditory Brainstem Response Test (ABR-TEST)

The mice (experimental groups G1 to G8) of Experimental Example <1-1> were anesthetized by intraperitoneal injection with 100 mg/kg of ketamine (Yuhan Kimberly, Seoul) and 10 mg/kg of xylazine hydrochloride (animal anesthetic; Bayer, Ansan), and the auditory brainstem response test for measuring auditory evoked potentials derived from the activity of the auditory nerve and central auditory pathway in response to transient sounds (auditory clicks or tone pips) was conducted. The wave V reference value of ABR was determined by gradually attenuating the sound intensity in 5 dB steps from 80 dB SPL until the wave V and the noise floor were indistinguishable from the recorded traces. Data management and auditory brainstem response test were conducted using the TDT RZ6/BioSigRZ system (Tucker Davis Technologies, Alachua, FL, USA) in a noise attenuation chamber with a built-in Faraday box.

Electrodes were disposed on the subcutaneous tissue of each mouse to collect data. The reference electrode on the same side as the stimulus was disposed axially on the pinna, the ground electrode was disposed on the opposite side, and the active electrode was disposed at the apex. An isothermal pad was used to maintain the body temperature of the mouse, and the ABR test was conducted every two weeks for stability of the experimental groups.

### <1-5> Superoxide Dismutase (SOD) Activity Test

The amount of SOD, an antioxidant enzyme that eliminates excess active oxygen accumulated in the body, was measured using blood collected from the retroorbital plexus of the mice (experimental groups G1 to G8) of Experimental Example <1-1>.

Specifically, the blood was reacted in an anticoagulant tube at 25° C. (room temperature) for 30 minutes, and then centrifuged at 2,000 × g for 15 minutes at 4° C. to prepare purified serum. The purified serum was stored at -80° C. for one month and diluted 1:5 with sample buffer before analysis, and then the amount of SOD in the body was measured using an SOD assay kit (No. 706002, Cayman Che., MI, USA). The SOD assay kit is a kit that uses tetrazolium salt to detect superoxide radicals generated by xanthine oxidase and hypoxanthine.

The SOD activity for each sample was calculated using the equation obtained from the linear regression of the standard curve, and the unit was defined as the amount of enzyme required when 50% of the radicals of SOD was substituted.

### <1-6> Histological Analysis

The mice (experimental groups G1 to G8) of Experimental Example <1-1> were sacrificed, and the cochlea was dissected and stained.

Specifically, an opening was made at the apex of the cochlear bone of the sacrificed mouse, and phosphate buffered saline at pH 7.4 containing 4% paraformaldehyde as a fixative was perfused at room temperature. The cochlea perfused with a fixative was immersed in Calci-Clear Rapid for 24 hours to demineralize, an OCT (Optimal Cutting Temperature) compound was inserted, and then the cochlea was cut into slices with a thickness of 2 to 5 µm using LEICA RM2145 (Leica Biosystems, Wetzlar, German). The cut cochlea was incubated in standard hematoxylin for 3 minutes and stained with eosin (H&E) for 60 seconds.

### <1-7> Immunostaining

The cochlear slice prepared in Experimental Example <1-6> was fixed and images of auditory hair cells were analyzed using a microscope.

Specifically, the cochlear slice was fixed on a gelatin-coated slide having a thickness of 5 µm with 4% paraformaldehyde for 15 minutes and then dried at room temperature. The slice was incubated with 5% normal goat serum at room temperature for 1 hour to prevent nonspecific labeling, and incubated with primary antibody MY07A (1:200, ab3481, Abeam, UK) at 4° C. for 1 hour. The incubated slice was washed with PBS three times for 5 minutes, incubated with a secondary antibody, goat antirabbit IgG H&L (Alexa Fluor® 488; 1:1000, abl50077, Abeam, UK) for 1 hour at room temperature, then washed again with PBS three times for 5 minutes, and fixed with a mounting solution containing DAPI (4′,6-diamidino-2-phenylindole).

The images of auditory hair cells of all the cochlear slices were analyzed using a confocal microscope (Carl Zeiss Microscopy GmbH, Jena, Germany) and ZEN lite ver.2.3 (ZEN lite, Jena, Germany) software.

### <1-8> SEM (Scanning Electron Microscopy) Analysis

The cochlear slice prepared in Experimental Example <1-6> was fixed and images of auditory hair cells were analyzed by scanning electron microscopy.

Specifically, the cochlear slice was primarily fixed with 2.5% glutaraldehyde at 4° C. for 2 hours, then washed with 0.1 M cacodylate buffer two times, and secondarily fixed with 1% osmium tetroxide (OsO₄). The fixed slice was dehydrated with 50%, 70%, 80%, 90% and 100% ethanol and then reacted with 3-methylbutyl acetate (isoamyl acetate, Hanawa, Japan) at room temperature for 15 minutes. The slice after the reaction was dried at room temperature for 15 minutes using hexamethyldisilazane (cat. 440191, Sigma-Aldrich, USA) and coated with gold, and the morphology of auditory hair cells was observed under a tabletop microscope (TM-1000, Hitachi Ltd., Tokyo, Japan).

### <1-9> RT-PCR (Real Time Polymerase Chain Reaction) Analysis

The mice (experimental groups G1 to G8) of Experimental Example <1-1> were sacrificed, and real-time PCR was conducted to analyze expression of genes associated with aging or damage in the liver, kidney and cochlea of the mice.

Specifically, total RNA was isolated using TRIzol (Thermo Fisher Scientific, San Diego, CA, USA) reagent.

To prepare mRNA samples, 2 µl of mRNA and a total of 8 µl of reverse transcriptase solution containing 1 µl of 10X enzyme mix, 2 µl of 5X enzyme reaction buffer, and 5 µl of nuclease-free water were mixed together. The prepared sample was reverse transcribed using SYBR Select master mix (Applied Biosystems, Calrsbad, CA, USA) according to the manufacturer’s protocol. Primer sequences used are shown in Table 10 below.

TABLE 10

| | | | |
|---|---|---|---|
| Target gene | Predicable Dysfunctional effect | F/R | 5′->3′ |
| Ednl | Cardiovascular complications | F | ACA CCG TCC TCT TCG TTT TG |
| | | R | GAG CTC CTT GGA AAG TCA CG |
| Slc24A4 | Anion transport dysfunction in auditory organ | F | TCA TTG CCT TTG GGA TAA GC |
| | | R | GGC AAC CAT CAC AAT CAC AG |
| Ucp2 | Mitochondria dysfunction | F | CTC AAA GCA GCC TCC AGA AC |
| | | R | ACA TCT GTG GCC TTG AAA CC |
| Kcnq4 | Non-syndromic | F | TGT TGG GAT CCG TGG TCT |
| | sensorineural hearing loss | | AT |
| | | R | GAG TTG GCA TCC TTC TCA GC |
| Myo7a | Non-syndromic hearing loss and deafness | F | GAC AAC TCT AGC CGC TTT GG |
| | | R | GAC ACG TGA CTT CTC CAG CA |
| Myo6 | Non-syndromic hearing loss and deafness | F | AGA CCA CTT CCG GCT CAC TA |
| | | R | TGG GTT GTC TCG TAG CAC AC |
| Cdh23 | Age-related hearing loss | F | ATG GAG AGC CCT CTG GAA AT |
| | | R | ACC CAC AAA GGC TGT ACT GG |
| ApoE | Age-related disorders, Vascular disease | F | GGT TCG AGC CAA TAG TGG AA |
| | | R | ATG GAT GTT GTT GCA GGA CA |
| 18 s | Ribosomal RNA | F | CAT TCG AAC GTC TGC CCT AT |
| | | R | GTT TCT CAG GCT CCC TCT CC |

As the RT-PCR process, a total of 10 µl of sample containing 1 µl of cDNA, 5 µl of premix, 1 µl of 10 pmol forward/reverse primers and 3 µl of nuclease-free water was subjected to preamplification at 90° C. for 3 minutes, 40 cycles of 95° C. for 10 seconds, and 60° C. for 60 seconds, and melting curve analysis was conducted. All of the processes were repeatedly performed, and normalization of mRNA expression levels was calculated using 18srRNA.

### <1-10> Statistical Analysis

Statistical analysis was performed using the SPSS statistical package version 21.0 (SPPS Inc., La Jolla, CA, USA).

Results of continuous variables were expressed as mean standard deviations of normally distributed variables, and mean hearing thresholds were compared by two-way ANOVA.

Statistically significant mRNA sensitivity and specificity were analyzed by the Mann-Whitney U-test using GraphPad PRISM version 5.0 (GraphPad Inc., La Jolla, CA, USA). A p-value of less than 0.05 was considered statistically significant.

### <Experimental Example 2> Experimental Results

### <2-1> Results of Group Phenotype Analysis on Aging

Changes in hair, body weight, and skin tissue of the mice (experimental groups G1 to G8) of Experimental Example <1-1> were checked.

As a result, in the hypoxia groups (G5 to G8), it has been confirmed that the hair of the mice has lost gloss and the color has changed to gray (see FIG. 3). Under different oxygen conditions, it has been confirmed that the effect of increasing body weight is insignificant as the body weight change in hypoxia after 3 months is 4 g (see b of FIG. 4), and the dermis thickness has been confirmed to increase by about 100 µm (see c of FIG. 4).

Under different feeding conditions, it has been confirmed that the body weight change of the normal diet groups after 3 months is less than 3 g but the body weight of HFD groups has increased by about 20 g, and it has been thus confirmed that HFD has the greatest effect of increasing body weight and fat layer (see a and b of FIG. 4).

Under different galactose injection conditions, the body weight change of the galactose injection groups in normoxia after 3 months is 8 to 10 g but the body weight change of the galactose injection groups in hypoxia is 3 to 6 g, and it has been thus confirmed that galactose injection is not related to the body weight change (see a and b of FIG. 4).

Table 11 below shows the changes in dermal and fat layer thicknesses after 3 months.

TABLE 11

| | WT (G1) | +GI (G2) | +HFD (G3) | +HFD, GI (G4) | +Hypoxic (G5) | +Hypoxic, HFD, GI (G8) |
|---|---|---|---|---|---|---|
| Dermis Thickness | 287.05 ±99.26 | 348.5 ±85.9 | 346.76 ±81.53 | 392.35 ±67.59 | 351.4 ±93.46 | 388.42 ±66.9 |
| Fat layer Thickness | 87.59 ±19.76 | 127.4 ±29.04 | 600.29 ±18.18 | 594.44 ±18.55 | 89.71 ±36.00 | 133.67 ±36.87 |

In conclusion, it has been confirmed that HFD most greatly affects the increase in the thickness of fat layer, a hypoxic condition greatly affects the increase in the thickness of dermis, and these affects the increase in deep wrinkles on the skin surface (see FIG. 4 and Table 11).

### <2-2> Analysis Results of Oxidative Stress in Serum

The amount of oxidative stress in serum was analyzed by analyzing the SOD activity test result in Experimental Example <1-5>.

As a result, it has been confirmed that oxidative stress increases in the hypoxia groups as the SOD value (G5, 0.6215 ± 0.048) of the hypoxia groups is higher than the SOD value (G1, 0.5311 + 0.019) of the control group after 3 months. In addition, it has been confirmed that oxidative stress has further increased under the dual and triple conditions including hypoxia than under the single condition of hypoxia as the SOD value (D7, 0.6453 + 0.055) of the hypoxia and HFD groups or the SOD value (D8, 0.5955 + 0.022) of the hypoxia, HFD and GI groups is higher than the SOD (G5, 0.6215 +0.048) value of the control group. However, there is no significant difference (about 0.05 to 0.1534) between the SOD value (G1, 0.5311 ± 0.019) of the control group and the SOD value (G3, 0.4901 + 0.011) of the HFD groups, the SOD value (D2, 0.5148 + 0.028) of the GI groups, and the SOD value (D4, 0.5572 + 0.050) of the HFD and GI groups after 3 months, and it has been thus confirmed that HFD and GI do not greatly affect oxidative stress (a difference of 0.04 or less may be an error value). On the other hand, it has been confirmed that SOD in hypoxia tends to increase compared to that in normoxia after 2 months, but SOD tends decreases after 3 months and oxidative stress tends to decrease in the normoxia groups (G1 to G3) (see FIG. 5).

Table 12 below shows the amount of SOD in serum confirmed.

TABLE 12

| GrouP | 0 M | 1 M | 2 M | 3 M |
|---|---|---|---|---|
| G1 | 0.6325 + 0.000 | 0.5873 + 0.005 | 0.5667 + 0.000 | 0.5311 ±0.019 |
| G2 | 0.5955 ± 0.017 | 0.5463 + 0.005 | 0.5216 + 0.005 | 0.5148 ±0.028 |
| G3 | 0.5586 ±0.011 | 0.5463 + 0.005 | 0.5298 + 0.005 | 0.4901 ±0.011 |
| G4 | 0.5873 + 0.005 | 0.5298 + 0.005 | 0.5011 ±0.011 | 0.5572 + 0.050 |
| G5 | 0.5873 + 0.029 | 0.5463 + 0.005 | 0.5463 + 0.005 | 0.6215 + 0.048 |
| G6 | 0.5914 ±0.000 | 0.5627 + 0.005 | 0.5463 + 0.005 | 0.5695 + 0.059 |
| G7 | 0.5996 + 0.000 | 0.5463 + 0.005 | 0.5914 ±0.000 | 0.6435 + 0.055 |
| G8 | 0.5750 ±0.011 | 0.5463 + 0.005 | 0.5380 + 0.005 | 0.5955 + 0.022 |

G1 = normoxia + NF, G2 = normoxia + NF + GI, G3 = normoxia + HDF, G4 = normoxia + HDF + GI
G5: hypoxia + NF, G6: hypoxia + NF + GI
G7: hypoxia + HFD, G8: hypoxia + HFD + GI

### <2-3> Results of Hearing Threshold Comparing Test

Hearing threshold values at frequencies of 4 to 32 kHz were analyzed in the tone-burst ABR of Experimental Example <1-4>.

As a result, it has been confirmed that changes in the hearing threshold of the control group is insignificant for 3 months but the hearing threshold of G8 (hypoxia + HFD + GI) has increased significantly from 35 dB to 67 dB at 8 kHz and from 31 dB to 70 dB at 16 kHz (see b of FIG. 6). In addition, it has been confirmed that the hearing threshold of G5 (hypoxic) has increased from 25 dB to 58 dB at 8 kHz and from 35 dB to 56 dB at 16 kHz (see a and e of FIG. 5). It has also been confirmed that the hearing threshold of G6 (hypoxia + GI) and G7 (hypoxia + HFD) has increased from 34 + 3.76 dB to 67 + 10.13 dB at 8 kHz and from 38 + 4.08 dB to 60 + 7.33 dB at 16 kHz (see g and h of FIG. 6).

However, it has been confirmed that there is no significant changes in the hearing thresholds of G2 (GI), G3 (HFD) and G4 (HFD + GI) (see c, d and f of FIG. 6).

Specifically, as a result of selecting three frequencies 8, 16, and 24 kHz with the greatest changes in the hearing threshold and analyzing the hearing threshold of each mice group, it has been confirmed that the hearing thresholds of G2 and G3 do not change significantly compared to that of the control group (see FIG. 7) but there is a significant change in the increase in hearing threshold of G5 after 2 months (see a of FIG. 8) under single conditions of G2 (GI), G3 (HFD) and G5 (hypoxia). It has also been confirmed that there is no change in G4 (GI + HFD) but the hearing threshold has significantly increased from 30 dB to 60 dB in G6 (hypoxia + GI) and G7 (hypoxia + HFD) after 1 month (see g, j and k of FIG. 8). In addition, it has been confirmed that the hearing threshold of G8 (hypoxia + GI + HFD) has significantly increased in a short period of time (see m and n of FIG. 8).

More specifically, as a result of analyzing the changed hearing thresholds by selecting 8 kHz to evaluate the determinant under single, dual and triple conditions, it has been confirmed that there is no significant change in the hearing threshold in GI (G2), HFD (G3) and GI + HFD (G4) (see a and b of FIG. 9) and the hearing threshold has increased within 2 months under the dual condition of HFD + hypoxia (G7) (see b and c of FIG. 9). In addition, it has been confirmed that the hearing threshold has increased after 2 months when hypoxic condition is included (G5 to G8), and it has been thus confirmed that a hypoxic condition and a HFD condition significantly affect the increase in hearing threshold (see a to c of FIG. 9).

In conclusion, it has been confirmed that the most effective method for changing the hearing threshold is a hypoxic condition, and that a synergy effect is exhibited when HFD is fed and/or galactose is injected in hypoxia.

### <2-4> Results of Hair Cell Histological Observation

The survival rate of hair cells was evaluated in the same manner as in Experimental Example <1-7>. The survival rate was evaluated by analyzing Myo7a, a major protein present in auditory hair cells.

As a result, three outer hair cells (OHC) and one inner hair cell (IHC) have been observed in 4-week-old mice (d of FIG. 9), and damage to hair cells has not been observed in the mice of G1 (control group) (e of FIG. 9).

It has also been confirmed that there has been almost no damage to cells in the GI groups (f of FIG. 9), moderate damage has occurred in the HFD groups (g of FIG. 6), and severe damage has occurred in the hypoxia groups (h of FIG. 9). In particular, it has been confirmed that damage to OHC is severe under the dual and triple conditions (i to k of FIG. 9).

It has been confirmed that the survival rate of hair cells is 80% or more under the single condition of HFD or GI and the survival rate is 50% or more under the dual condition of HFD and GI, but the survival rate decreases to less than 20% when a hypoxic condition is included, and it has been thus confirmed that oxidative stress caused by hypoxia damages hair cells and causes hearing loss.

In addition, in order to further analyze damage due to oxidative stress, damage to stereocilia of hair cells was examined by analyzing the SEM images analyzed in Experimental Example <1-8>.

As a result, it has been confirmed that intact OHC and IHC lines can be observed in 4-week-old mice (a and d of FIG. 10) but OHC has been damaged by oxidative stress in mice under a hypoxic condition (b and e of FIG. 10). In the hypoxia, HFD and GI groups, it has been observed that most stereocilia of hair cells have disappeared and it has been confirmed that most OHCs have been damaged, and it has been confirmed that auditory hair cells are damaged by oxidative stress due to a hypoxia, HFD or GI condition (see c and f of FIG. 10).

### <2-5> Results of Age-Related Factor Phenotype Analysis of Cochlear

By analyzing the results of PCR conducted in Experimental Example <1-9>, changes in the age-related factor phenotype of the cochlea were analyzed.

As a result, it has been confirmed that the expression of Edn1, Slc24A4, Ucp2, Kcnq4, Myo7a, Myo6, Cdh23, and ApoE, genes associated with aging of or damage to the auditory organ, has significantly increased under a triple condition of hypoxia, HFD, and GI (see FIG. 11).

Specifically, it has been confirmed that ion channel-related genes in the auditory organ, such as Slc26a4 and Kcnq4, have been overexpressed (b and d of FIG. 11) and Ucp2 expressed in mitochondria dysfunction has significantly increased compared to other genes (c of FIG. 11). It has also been confirmed that the expression of Myo7a and Myo6 has significantly increased by damage to the auditory organ (e and f of FIG. 11) and the expression of Cdh23, which is the most expressed gene in the aging process of the auditory organ, has also increased (a of FIG. 7). However, it has been confirmed that the expression level of ApoE is similar to that under the triple condition even under the single condition of hypoxia or HFD in addition to the triple condition (h of FIG. 11).

In conclusion, it has been confirmed that the expression of aging factors significantly increases under dual and triple conditions of hypoxia, HFD, and GI than under the single conditions thereof.

### INDUSTRIAL APPLICABILITY

The present invention relates to an animal model for age-related hearing loss (ARHL, presbycusis) and a method for preparing the same, particularly the present invention can be usefully utilized to prepare and provide an animal model for age-related hearing loss since the auditory organ is aged and damaged in a short period of time when an animal is fed with a high fat diet and injected with galactose in intermittent hypoxia.

1. A method for preparing an animal model for age-related hearing loss, the method comprising:
(1) selecting a laboratory animal with normal hearing among non-human animals;
(2) introducing the laboratory animal selected in step (1) into a chamber for creating a hypoxic condition;
(3) creating an intermittent hypoxic condition in the chamber of step (2); and
(4) feeding a high fat diet or injecting galactose to the animal of step (3).

2. The method for preparing an animal model for age-related hearing loss according to claim 1,
wherein the laboratory animal of step (1) is one or more non-human animals selected from the group consisting of rats, mice, rat gerbils, guinea pigs, monkeys, dogs, cats, rabbits, cows, sheep, pigs and goats.

3. The method for preparing an animal model for age-related hearing loss according to claim 1,
wherein the intermittent hypoxic condition in step (3) is a condition in which hypoxia having an oxygen concentration of 0% to 10% for 8 to 16 hours and normoxia having an oxygen concentration of 15% to 25% for 8 to 16 hours are alternately created.

4. The method for preparing an animal model for age-related hearing loss according to claim 1,
wherein the intermittent hypoxic condition in step (3) is a condition in which hypoxia having an oxygen concentration of 0% to 5% for 12 hours and normoxia having an oxygen concentration of 15% to 25% for 12 hours are alternately created.

5. The method for preparing an animal model for age-related hearing loss according to claim 1,
wherein feeding of a high fat meal and injection of galactose are performed in parallel in step (4).

6. The method for preparing an animal model for age-related hearing loss according to claim 1,
wherein the high fat diet in step (4) is a food containing vitamins and having a fat content of 25% to 60%.

7. The method for preparing an animal model for age-related hearing loss according to claim 1,
wherein galactose in step (4) is 100 to 800 mg per unit body weight (kg) of the animal.

8. The method for preparing an animal model for age-related hearing loss according to claim 1,
wherein galactose in step (4) is 250 to 600 mg per unit body weight (kg) of the animal.

9. The method for preparing an animal model for age-related hearing loss according to claim 1,
wherein the method is carried out over a period of 4 to 16 weeks.

10. The method for preparing an animal model for age-related hearing loss according to claim 1,
wherein the method is carried out over a period of 8 to 12 weeks.

11. The method for preparing an animal model for age-related hearing loss according to claim 1,
wherein the age-related hearing loss is one or more kinds of hearing loss selected from the group consisting of sensory presbycusis, neural presbycusis, metabolic presbycusis, cochlear conductive presbycusis, mixed presbycusis, and intermediate presbycusis.

12. The method for preparing an animal model for age-related hearing loss according to claim 1,
wherein hearing loss in the animal model for age-related hearing loss is expressed by one or more effects selected from the group consisting of fat accumulation, increased oxidative stress, induction of mitochondria dysfunction, accelerated aging, auditory organ aging and damage-associated gene expression, and auditory hair cell death effect.

13. The method for preparing an animal model for age-related hearing loss according to claim 12,
wherein the expressed gene is one or more genes selected from the group consisting of Edn1, a gene associated with cardiovascular complication, Slc24A4, an ion channel-related gene in the auditory organ, Ucp2, a gene associated with mitochondria dysfunction, Kcnq4, Myo7a, and Myo6, genes associated with non-syndromic hearing loss, Cdh23, a gene associated with age-related hearing loss, and ApoE, a gene associated with age-related disorders and vascular diseases.

14. The method for preparing an animal model for age-related hearing loss according to claim 1,
wherein the age-related hearing loss has a hearing threshold of 30 dB or more.

15. The method for preparing an animal model for age-related hearing loss according to claim 1,
wherein the age-related hearing loss is maintained chronically.

16. The method for preparing an animal model for age-related hearing loss according to claim 1,
wherein the age-related hearing loss is maintained for 3 months or more.

17. An animal model for age-related hearing loss prepared by the preparation method according to claim 1.

18. A method for screening a drug for age-related hearing loss, comprising:
(a) administering a drug candidate substance for age-related hearing loss to the animal model for age-related hearing loss according to claim 17;
(b) measuring a hearing threshold of the animal model to which the candidate substance of step (a) has been administered; and
(c) comparing the hearing threshold of the animal model of step (b) with a hearing threshold of an animal model not administered with the candidate substance, and selecting the candidate substance as a drug for age-related hearing loss when the hearing threshold is decreased.
